SEQUENCE LISTING (32 sequences)

SEQ ID NO: 1 - DNA, 20 bp, Artificial Sequence
  Oligonucleotide
cggcgaatgctgctcctgtc

SEQ ID NO: 2 - DNA, 20 bp, Artificial Sequence
  Oligonucleotide
cgagatggcctggggacgaa

SEQ ID NO: 3 - DNA, 20 bp, Artificial Sequence
  Oligonucleotide
gctgaagctgccctcgagga

SEQ ID NO: 4 - DNA, 20 bp, Artificial Sequence
  Oligonucleotide
cgagatggcctggggacgaa

SEQ ID NO: 5 - DNA, 21 bp, Artificial Sequence
  Oligonucleotide
gttttcgtccccaggccatct

SEQ ID NO: 6 - DNA, 19 bp, Artificial Sequence
  Oligonucleotide
ggggaaagctgccaccgtg

SEQ ID NO: 7 - DNA, 20 bp, Artificial Sequence
  Oligonucleotide
gttttcgtccccaggccatc

SEQ ID NO: 8 - DNA, 20 bp, Artificial Sequence
  Oligonucleotide
ccaacatcgccaagggacct

SEQ ID NO: 9 - DNA, 20 bp, Artificial Sequence
  Oligonucleotide
gctgaagctgccctcgagga

SEQ ID NO: 10 - DNA, 20 bp, Artificial Sequence
  Oligonucleotide
accagtgcctgaagaggttt

SEQ ID NO: 11 - DNA, 21 bp, Artificial Sequence
  Oligonucleotide
gtctccaccattcgagtctga

SEQ ID NO: 12 - DNA, 21 bp, Artificial Sequence
  Oligonucleotide
accagtgcctgaagaggtttt

SEQ ID NO: 13 - DNA, 19 bp, Artificial Sequence
  Oligonucleotide
gaatgctgctcctgtccaa

SEQ ID NO: 14 - DNA, 21 bp, Artificial Sequence
  Oligonucleotide
accagtgcctgaagaggtttt

SEQ ID NO: 15 - DNA, 20 bp, Artificial Sequence
  Oligonucleotide
cactggtgccgaggacccta

SEQ ID NO: 16 - DNA, 20 bp, Artificial Sequence
  Oligonucleotide
gacctgcgtttccgagatgg

SEQ ID NO: 17 - DNA, 16 bp, Artificial Sequence
  Oligonucleotide
ctgcacaacgtggttt

SEQ ID NO: 18 - DNA, 17 bp, Artificial Sequence
  Oligonucleotide
gtgcctgaagaggtttt

SEQ ID NO: 19 - DNA, 21 bp, Artificial Sequence
  Oligonucleotide
tgccgaggaccctaggccatc

SEQ ID NO: 20 - DNA, 21 bp, Artificial Sequence
  Oligonucleotide
ccaacatcgccaagggacctg

SEQ ID NO: 21 - DNA, 21 bp, Artificial Sequence
  Oligonucleotide
tgccgaggaccctaggccatc

SEQ ID NO: 22 - DNA, 21 bp, Artificial Sequence
  Oligonucleotide
tcacattcccaacatcgccaa

SEQ ID NO: 23 - DNA, 20 bp, Artificial Sequence
  Oligonucleotide
aggaccctaggccatctcgg

SEQ ID NO: 24 - DNA, 19 bp, Artificial Sequence
  Oligonucleotide
ccaacatcgccaagggacc

SEQ ID NO: 25 - DNA, 21 bp, Artificial Sequence
  Oligonucleotide
gcactggtgccgaggacccta

SEQ ID NO: 26 - DNA, 20 bp, Artificial Sequence
  Oligonucleotide
ggcacggccaccgtcacatt

SEQ ID NO: 27 - DNA, 20 bp, Artificial Sequence
  Oligonucleotide
tgccgaggaccctaggccat

SEQ ID NO: 28 - DNA, 20 bp, Artificial Sequence
  Oligonucleotide
ggcacggccaccgtcacatt

SEQ ID NO: 29 - DNA, 20 bp, Artificial Sequence
  Oligonucleotide
tgccgaggaccctaggccat

SEQ ID NO: 30 - DNA, 20 bp, Artificial Sequence
  Oligonucleotide
cggccaccgtcacattccca

SEQ ID NO: 31 - DNA, 3575 bp, Artificial Sequence
  Genetically-engineered plasmid
gaaagtcctctccactgactgtagcctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctccttcctcttccagcccttcctctttcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagct

SEQ ID NO: 32 - DNA, 3611 bp, Artificial Sequence
  Genetically-engineered plasmid
gaaagtcctctccactgactgtagcctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctccttcctcttccagcccttcctctttcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccagaaaaacctcttcaggcactggtgccgaggaccctaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagct

(19) United States
(12) Patent Application Publication
(10) Pub. No.: US 2012/0183966 A1
(43) Pub. Date: Jul. 19, 2012

(54) DETECTION AND QUANTITATION OF INSULIN RECEPTOR ALPHA ISOFORM OR BETA ISOFORM

(76) Inventors: Sean C. Harrington, Rochester, MN (US); Paul Haluska, Rochester, MN (US)

(21) Appl. No.: 13/302,358

(22) Filed: Nov. 22, 2011

Related U.S. Application Data
(60) Provisional application No. 61/416,009, filed on Nov. 22, 2010.

(51) Int. Cl. C12Q 1/68

(52) U.S. Cl. 435/6.12

(57) ABSTRACT

Provided are methods and materials for quantitatively detecting the insulin receptor alpha isoform and the insulin receptor beta isoform.

### CROSS REFERENCE TO RELATED APPLICATIONS

This application claims benefit under 35 U.S.C. 119(e) to U.S. Application No. 61/416,009 filed Nov. 22, 2010, the disclosure of which is incorporated herein in it's entirely.

### TECHNICAL FIELD

This disclosure generally relates to quantitative detection of insulin receptor isotype alpha and insulin receptor isotype beta.

### BACKGROUND

The insulin receptor is important in cancer. Understanding how the insulin receptor functions in cancer is complicated by the fact that it is expressed in two different forms that have very different functions. One form, referred to as isoform beta, appears to be involved in telling cells when to remove and store sugar from their environment. The other form, referred to as isoform alpha, appears to be involved in helping cancer cells grow. Currently, there are no standard methods for telling one insulin receptor isoform from the other in tumors or other tissues. This disclosure describes methods to determine which receptor isoform is present and to determine how much of each receptor isoform is present in tumors or other tissues.

### SUMMARY

Methods and materials are provided for quantitatively detecting the insulin receptor alpha isoform and the insulin receptor beta isoform.

In one aspect, methods of determining the amount of insulin receptor alpha in a sample are provided. Such methods typically include the steps of a) amplifying an insulin receptor alpha sequence in a sample in which cDNAs have been produced from reverse transcription of the RNA using a pair of insulin receptor alpha primers to produce an insulin receptor alpha amplification product, wherein the pair of insulin receptor alpha primers is selected from the group consisting of SEQ ID NOs: 1 and 2; SEQ ID NOs: 3 and 4; SEQ ID NOs: 5 and 6; and SEQ ID NOs: 7 and 8; and b) determining the amount of the insulin receptor alpha amplification product.

In another aspect, methods of detecting the amount of insulin receptor beta in a sample are provided. Such methods typically include the steps of a) amplifying an insulin receptor beta sequence in a sample in which cDNAs have been produced from reverse transcription of the RNA using a pair of insulin receptor beta primers to produce an insulin receptor beta amplification product, wherein the pair of insulin receptor beta primers is selected from the group consisting of SEQ ID NOs: 9 and 10; SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; and SEQ ID NOs: 29 and 30; and b) determining the amount of the insulin receptor beta amplification product.

In still another aspect, methods of determining the amount of insulin receptor alpha in a sample are provided. Such methods can include the steps of a) obtaining RNAs from a sample; b) reverse transcribing the RNAs from step a) to produce cDNAs; c) amplifying an insulin receptor alpha sequence in the cDNAs from step b) using a pair of insulin receptor alpha primers to produce an insulin receptor alpha amplification product, wherein the pair of insulin receptor alpha primers is selected from the group consisting of SEQ ID NOs: 1 and 2; SEQ ID NOs: 3 and 4; SEQ ID NOs: 5 and 6; and SEQ ID NOs: 7 and 8; and d) determining the amount of the insulin receptor alpha amplification product.

In certain embodiments, the sample is a biopsy (e.g., a tumor biopsy). For example, the sample can be from a patient that has cancer. In some embodiments, the pair of primers is SEQ ID NOs:7 and 8. In some embodiments, the determining step involves the use of fluorescently-labeled hybridization probes and occurs in real-time with the amplifying step.

In one aspect, an article of manufacture is provided. Such an article of manufacture (e.g., a kit) can include a pair of primers for detecting the amount of insulin receptor alpha in a sample, wherein the pair of primers is selected from the group consisting of SEQ ID NOs: 1 and 2; SEQ ID NOs: 3 and 4; SEQ ID NOs: 5 and 6; and SEQ ID NOs: 7 and 8. In certain embodiments, the pair of primers is SEQ ID NOs: 7 and 8. In some embodiments, an article of manufacture further can include a pair of insulin receptor alpha probes. In some embodiments, an article of manufacture further can include a donor fluorophore and an acceptor fluorophore.

In yet another aspect, methods of detecting the amount of insulin receptor beta in a sample are provided. Such methods can include the steps of a) obtaining RNAs from a sample; b) reverse transcribing the RNAs from step a) to produce cDNAs; c) amplifying an insulin receptor beta sequence in the cDNAs from step b) using a pair of insulin receptor beta primers to produce an insulin receptor beta amplification product, wherein the pair of insulin receptor beta primers is selected from the group consisting of SEQ ID NOs: 9 and 10; SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; and SEQ ID NOs: 29 and 30; and d) determining the amount of the insulin receptor beta amplification product.

In certain embodiments, the sample is a biopsy (e.g., a tumor biopsy). For example, the sample can be from a patient that has cancer. In some embodiments, the pair of primers is SEQ ID NOs:15 and 16. In some embodiments, the determining step involves the use of fluorescently-labeled hybridization probes and occurs in real-time with the amplifying step.

In one aspect, an article of manufacture is provided. Such an article of manufacture (e.g., a kit) can include a pair of primers for detecting the amount of insulin receptor beta in a sample, wherein the pair of primers is selected from the group consisting of SEQ ID NOs: 9 and 10; SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; and SEQ ID NOs: 29 and 30. In certain embodiments, the pair of primers is SEQ ID NOs: 15 and 16. In some embodiment, an article of manufacture further can include a pair of insulin receptor alpha probes. In some embodiments, an article of manufacture further can include a donor fluorophore and an acceptor fluorophore.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A is a schematic demonstrating the location of binding of a number of different primers within the insulin receptor alpha sequence; FIG. 1B is a schematic indicating the position of the insulin receptor alpha sequence in the BHpUCminusMCS plasmid and the position of the insulin receptor alpha (IR-A) primer set #4 within the insulin receptor alpha sequence; and FIG. 1C is an image of insulin receptor alpha primer product generated using dilutions of insulin receptor alpha plasmid. * 50 bp product resulting from amplification with the IR-A primer set #4.

FIG. 2A is a schematic demonstrating the location of binding of a number of primers within the insulin receptor beta sequence; FIG. 2B is a schematic indicating the position of the insulin receptor beta sequence in the BHpUCminusMCS plasmid and the position of the insulin receptor beta (IR-B) primer set #4 within the insulin receptor beta sequence; and FIG. 2C is an image of insulin receptor beta primer products generated using dilutions of insulin receptor beta plasmid. * 42 bp product resulting from amplification with the IR-B primer set #4.

FIG. 3 is the sequence of the insulin receptor alpha construct (SEQ ID NO:31) in the BHpUCminusMCS construct.

FIG. 4 is the sequence of the insulin receptor beta construct (SEQ ID NO:32) in the BHpUCminusMCS construct.

FIG. 5 is a graph showing the specificity and cross-reactivity of the insulin receptor alpha primers with the insulin receptor alpha (InsR-A) or beta (InsR-B) plasmids.

FIG. 6 is a graph showing the specificity and cross-reactivity of the insulin receptor beta primers with the insulin receptor beta (InsR-B) or alpha (InsR-A) plasmids.

FIG. 7 is a graph showing that quantification and recovery of insulin receptor alpha (A) is independent of insulin receptor beta (B).

FIG. 8 is a graph showing that quantification and recovery of insulin receptor beta (B) is independent of insulin receptor alpha (A).

FIG. 9 is a graph that demonstrates the limit of detection for insulin receptor alpha (circle) and beta (square) primer sets #4.

FIG. 10 shows the range of values reported by the insulin receptor alpha (IR-A) and beta (IR-B) primer sets using clinical formalin fixed paraffin embedded (FFPE) tumor source material.

### DETAILED DESCRIPTION

A quantitative PCR (qPCR) assay for determining the amount of insulin receptor alpha and insulin receptor beta in a sample is described herein. This disclosure provides insulin receptor alpha primers for determining the amount of insulin receptor alpha in a sample and insulin receptor beta primers for determining the amount of insulin receptor beta in a sample. This disclosure also provides articles of manufacture containing such insulin receptor alpha primers and insulin receptor beta primers.

### Insulin Receptor Nucleic Acids and Oligonucleotides

The human IR exists in two isoforms, isoform A (IR-A) and isoform B (IR-B). Alternative splicing of a small exon (exon 11) of the IR gene results in two transcripts, in which 36 additional nucleotides encoding 12 amino acids (residues 718 to 729) at the carboxyl terminus of the receptor alpha-subunit are either excluded (Ex11− or type A insulin receptor (IR-A)) or included (Ex11+ or type B insulin receptor (IR-B)). The relative expression of the two isoforms varies in a tissue-specific manner.

This document provides methods to determine the amount of insulin receptor alpha and insulin receptor beta by PCR amplification. The sequence of the human insulin receptor alpha mRNA can be found, for example, at GenBank Accession No. NM_{—}001079817, while the sequence of the human insulin receptor beta mRNA can be found, for example, at GenBank Accession No. NM_{—}000208. Specifically, this disclosure provides primers to specifically amplify and quantitate insulin receptor isoform alpha and insulin receptor isoform beta. Nucleic acids other than those exemplified herein also can be used to determine the amount of insulin receptor alpha and insulin receptor beta in a sample. Insulin receptor alpha and insulin receptor beta nucleic acids other than those exemplified herein (e.g., functional variants) can be evaluated (e.g., for specificity and/or sensitivity) by those of skill in the art using routine methods such as, but not limited to, the amplification methods exemplified herein. Representative functional variants, for example, include deletions of, insertions in, and/or substitutions in the insulin receptor nucleic acids disclosed herein.

Primers that amplify an insulin receptor alpha or beta nucleic acid can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 (e.g., 15, 16, 17, 18, 19, 20, 21, 22, 23, 24, 25, 26, 27, 28, 29, or 30) nucleotides in length.

Constructs or vectors including an insulin receptor alpha or insulin receptor beta nucleic acid (e.g., SEQ ID NOs: 31 and 32) are provided. Constructs can be used, for example, as control template nucleic acid molecules. Constructs suitable for use according to the methods provided herein are commercially available and/or produced by recombinant nucleic acid technology methods routine in the art. Insulin receptor alpha or insulin receptor beta nucleic acids can be obtained, for example, by chemical synthesis, direct cloning from cells, or by PCR amplification. An insulin receptor nucleic acid molecule or fragment thereof can be operably linked to a promoter or other regulatory element such as an enhancer sequence, a response element, or an inducible element that modulates expression of the insulin receptor nucleic acid. As used herein, “operably linking” refers to connecting a promoter and/or other regulatory elements to an insulin receptor nucleic acid in such a way as to permit and/or regulate expression of the insulin receptor nucleic acid. Constructs suitable for use according to the methods provided herein typically include, in addition to insulin receptor alpha or beta nucleic acid molecules, sequences encoding a selectable marker (e.g., an antibiotic resistance gene) for selecting desired constructs and/or transformants, and an origin of replication. The choice of constructs usually depends upon several factors, including, but not limited to, the choice of host cells, replication efficiency, selectability, inducibility, and the ease of recovery.

Constructs containing KPC nucleic acid molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct of the invention can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

### Amplification of Nucleic Acids by Polymerase Chain Reaction (PCR) and Real Time PCR

U.S. Pat. Nos. 4,683,202, 4,683,195, 4,800,159, and 4,965,188 disclose conventional polymerase chain reaction (PCR) techniques. PCR typically employs two oligonucleotide primers that bind to a selected nucleic acid template (e.g., DNA or RNA). Primers are discussed above and generally refer to oligonucleotides capable of acting as a point of initiation of nucleic acid synthesis within insulin receptor nucleic acid sequences. A primer can be produced synthetically, or it can be purified from a restriction digest by conventional methods. The primer is preferably single-stranded for maximum efficiency in amplification, but the primer can be double-stranded. Double-stranded primers are first denatured, i.e., treated to separate the strands. One method of denaturing double stranded nucleic acids is by heating.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 10 seconds to 4 minutes (e.g., 30 seconds, or 1 minute to 2 minutes 30 seconds, or 1 minute 30 seconds).

If the double-stranded template nucleic acid is denatured by heat, the reaction mixture is allowed to cool to a temperature that promotes annealing of each primer to its target sequence on the insulin receptor nucleic acid. The temperature for annealing is usually from about 35° C. to about 65° C. (e.g., about 40° C. to about 60° C.; about 45° C. to about 50° C.). Annealing times can be from about 10 seconds to about 1 minute (e.g., about 20 seconds to about 50 seconds; about 30 seconds to about 40 seconds). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 50° to 80° C. (e.g., about 65° C. to about 75° C.; about 70° C.). Extension times can be from about 10 seconds to about 5 minutes (e.g., about 30 seconds to about 4 minutes; about 1 minute to about 3 minutes; about 1 minute 30 seconds to about 2 minutes).

The steps of denaturing a template nucleic acid, annealing primers to the template nucleic acid at a temperature that is below the melting temperatures of the primers, and enzymatically elongating from the primers to generate an amplification product are typically referred to collectively as “amplification”. Amplifying can be described as the process of synthesizing nucleic acid molecules that are complementary to one or both strands of a template nucleic acid molecule (e.g., insulin receptor nucleic acids). Amplification typically requires the presence of deoxyribonucleoside triphosphates, a DNA polymerase enzyme (e.g., PLATINUM® Taq) and an appropriate buffer and/or co-factors for optimal activity of the polymerase enzyme (e.g., MgCl₂ and/or KCl).

PCR assays can employ insulin receptor DNA. The template nucleic acid need not be purified; it may be a minor fraction of a complex mixture, such as insulin receptor nucleic acid contained in human cells. Insulin receptor nucleic acids may be extracted from a biological sample by routine techniques such as those described in Sambrook et al., Molecular Cloning: A Laboratory Manual, 1982, Cold Spring Harbor Laboratory, Cold Spring Harbor, N.Y. Generally, the oligonucleotide primers (e.g., SEQ ID NOs:1-30) are combined with PCR reagents under reaction conditions that induce primer extension.

The newly synthesized strands form a double-stranded molecule that can be used in the succeeding steps of the reaction. The steps of strand separation, annealing, and elongation are repeated as often as needed to produce a reliably detectable amount of insulin receptor alpha and insulin receptor beta amplification products. Generally, the steps are repeated at least about 20 times, but may be repeated as many as 40, 60, or even 100 times.

Real time PCR refers to the concurrent detection of the amplification products as they are produced. Real time PCR typically uses FRET technology (see, for example, U.S. Pat. Nos. 4,996,143, 5,565,322, 5,849,489, and 6,162,603), which is based on a concept that, when a donor and a corresponding acceptor fluorescent moiety are positioned within a certain distance of each other and excited, energy transfer takes place between the two fluorescent moieties that can be visualized or otherwise detected and/or quantitated. In real time PCR, one probe containing both fluorescent moieties or two probes, each containing a fluorescent moiety, can hybridize to an amplification product. Upon hybridization of the oligonucleotide probe(s) to the amplification product and excitation, a FRET signal is generated. Excitation to initiate energy transfer can be carried out with an argon ion laser, a high intensity mercury (Hg) arc lamp, a fiber optic light source, or other high intensity light source appropriately filtered for excitation in the desired range.

Designing oligonucleotides to be used as hybridization probes can be performed in a manner similar to the design of primers, although the members of a pair of probes preferably anneal to an amplification product within no more than 5 nucleotides of each other on the same strand such that FRET can occur (e.g., within no more than 1, 2, 3, or 4 nucleotides of each other). This minimal degree of separation typically brings the respective fluorescent moieties into sufficient proximity such that FRET occurs. It is to be understood, however, that other separation distances (e.g., 6 or more nucleotides) are possible provided the fluorescent moieties are appropriately positioned relative to each other (for example, with a linker arm) such that FRET can occur. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 (e.g., 15, 16, 17, 18, 19, 20, 21, 22, 23, 24, 25, 26, 27, 28, 29, or 30) nucleotides in length. In addition, hybridization temperatures for the detection component in real time PCR reactions can range from about 35° C. to about 65° C. (e.g., about 40° C. to about 60° C.; about 45° C. to about 55° C.; about 50° C.) for about 10 seconds to about 1 minute (e.g., about 20 seconds to about 50 seconds; about 30 seconds to about 40 seconds).

With respect to donor and corresponding acceptor fluorescent moieties, “corresponding” refers to an acceptor fluorescent moiety having an emission spectrum that overlaps the excitation spectrum of the donor fluorescent moiety. Representative donor fluorescent moieties that can be used with various acceptor fluorescent moieties in FRET technology include fluorescein, Lucifer Yellow, B-phycoerythrin, and 9-acridineisothiocyanate. Representative acceptor fluorescent moieties, depending upon the donor fluorescent moiety used, include LCT™-Red 640, LCT™-Red 705, Cy5, tetramethyl rhodamine isothiocyanate, erythrosine isothiocyanate, and fluorescein. Donor and acceptor fluorescent moieties can be obtained from, for example, Molecular Probes (Junction City, Oreg.) or Sigma Chemical Co. (St. Louis, Mo.). The donor and acceptor fluorescent moieties can be attached to the appropriate probe oligonucleotide via a linker (e.g., thiourea linkers or amide linkers) using methods routine in the art. Analysis of the fluorescent signal generated can be carried out using, for example, a photon counting epifluorescent microscope system (containing the appropriate dichroic mirror and filters for monitoring fluorescent emission at the particular range), a photon counting photomultiplier system or a fluorometer.

Melting curve analysis is an additional step that can be included in a cycling profile. Melting curve analysis is based on the fact that DNA melts at a characteristic temperature, i.e., the melting temperature (Tm), which is defined as the temperature at which half of the DNA duplexes have separated into single strands. By detecting the temperature at which signal is lost, the melting temperature of probes can be determined. Similarly, by detecting the temperature at which signal is generated, the annealing temperature of probes can be determined. The melting temperature(s) of the probes (e.g., insulin receptor alpha probe(s), insulin receptor beta probe(s)) from the insulin receptor alpha or beta amplification product can confirm the presence of the particular insulin receptor isoform in the sample.

Any number of samples can be used in the disclosed methods. Representative samples that can be used in the methods described herein include, without limitation, blood, saliva, buccal mucosal cells, ascities, or cancer cells. Cancer cells from solid tumors, in pleural fluid, or in urine. In one embodiment, the biological sample is a biopsy. Collection and storage methods of samples (e.g., biological samples) are known to those skilled in the art. Samples can be processed (e.g., by nucleic acid extraction methods and/or kits known in the art) to release insulin receptor nucleic acid or in some cases, the biological sample can be contacted directly with the PCR reaction components and the appropriate oligonucleotides.

Within each thermocycler run, control samples can be cycled as well. Positive controls can utilize amplification of a control template (e.g., nucleic acid other than insulin receptor sequences) using, for example, control primers (and control probes, if real time PCR is being used) and/or amplification of, for example, a construct containing insulin receptor alpha and/or beta nucleic acids. Such a control construct can be amplified internally (e.g., within each test sample) or as a separate sample run side-by-side with the test samples. Each thermocycler run should also include a negative control that, for example, lacks any insulin receptor template DNA. Such controls are indicators of the success or failure of the amplification, hybridization and/or FRET reaction.

### Articles of Manufacture/Kits

This disclosure also provides for articles of manufacture that can be used to determine the amount of insulin receptor alpha or insulin receptor beta. An article of manufacture as provided herein can include insulin receptor alpha primers used to detect insulin receptor alpha nucleic acids and/or insulin receptor beta primers used to detect insulin receptor beta nucleic acids, together with suitable packaging materials. Representative insulin receptor alpha primers include, for example, SEQ ID NOs:1 and 2, 3 and 4, 5 and 6, or 7 and 8), and representative insulin receptor beta primers include, for example, SEQ ID NOs: 9 and 10, 11 and 12, 13 and 14, 15 and 16, 17 and 18, 19 and 20, 21 and 22, 23 and 24, 25 and 26, 27 and 28, and 29 and 30.

Articles of manufacture provided herein also can include one or more hybridization probes and may further include one or more fluorescent moieties for use in real time PCR. For example, an article of manufacture may include a donor fluorescent moiety for labeling one of the probes and an acceptor fluorescent moiety for labeling the same or another probe. Examples of suitable FRET donor fluorescent moieties and corresponding acceptor fluorescent moieties are well known in the art.

Articles of manufacture provided herein also can contain a package insert or package label having instructions thereon for using the insulin receptor alpha primers and/or insulin receptor beta primers to determine the amount of the corresponding insulin receptor nucleic acid in a sample. Articles of manufacture may additionally include reagents for carrying out the methods disclosed herein (e.g., buffers, polymerase enzymes, or co-factors). Such reagents may be specific for one of the commercially available PCR or real time PCR instruments.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Obtaining RNA

Tissue Preservation in RNA-Later

50 mm³ of freshly isolated tissue was placed in 700 μL of RNA-later. Sample were stored overnight at 4° C. before being stored at −20° C. Alternatively, RNA-later-preserved samples were stored up to 30 days at 4° C.

Preparation of RNA-Later-Preserved Tissue for RNA Isolation

The volume of RLT lysis buffer needed was calculated. (350 μL/sample) and the calculated volume was supplemented with 10 μL BME per mL. 350 μL of prepared RLT lysis buffer was added to one 1.7 mL microcentrifuge tube per sample, and placed on wet ice.

Tissue was removed from storage and placed on wet ice. Tissue was removed from the RNA-later storage tube and placed on a paper towel. A 20 mm³ piece was cut from the sample and the remaining tissue was placed back into the storage tube and placed on ice. A second paper towel was placed over the 20 mm³ sample and compressed using curved forceps. The sample was moved to a different location on the paper towel and the compression was repeated. This procedure was repeated until no more RNA-later buffer could be visibly wicked from the sample. Using fine scissors, the flattened tissue was cut into thin strips, which were then bisected with fine scissors at least once before the tissue fragments were transferred into 350 μL of prepared RLT lysis buffer. The sample was placed on ice.

Using a Pellet Pestle motor unit equipped with a disposable RNase-free sample pestle, each sample was homogenized until tissue fragments were no longer visible. Genomic DNA in each sample was sheered with a syringe equipped with a 22-guage needle and clarified by centrifuging for 5 minutes at maximum speed. The RLT supernatant was transferred into a new 1.7 mL microcentrifuge tube, being careful to leave behind the pellet.

RNA Isolation from Cells and Prepared Tissue

RNA isolation was performed using the Qiagen RNeasy Mini Kit protocol supplemented with the optional on-column DNase digestion. Total RNA was quantified using a Nanodrop 2000c spectrophotometer. RNA samples with a 260/280 ratio greater than 1.8 was considered acceptable; RNA samples with a ratio of less than 1.8 were re-purified.

700 ng of total RNA was reverse transcribed using ABI's High capacity RNA-to-cDNA kit in a reaction volume of 25 μL. The cDNA was diluted with 100 μL of RNase DNase-free water and stored at −20° C. until needed.

### Example 2

### Insulin Receptor Primer Sets

Insulin Receptor A Primer Sets

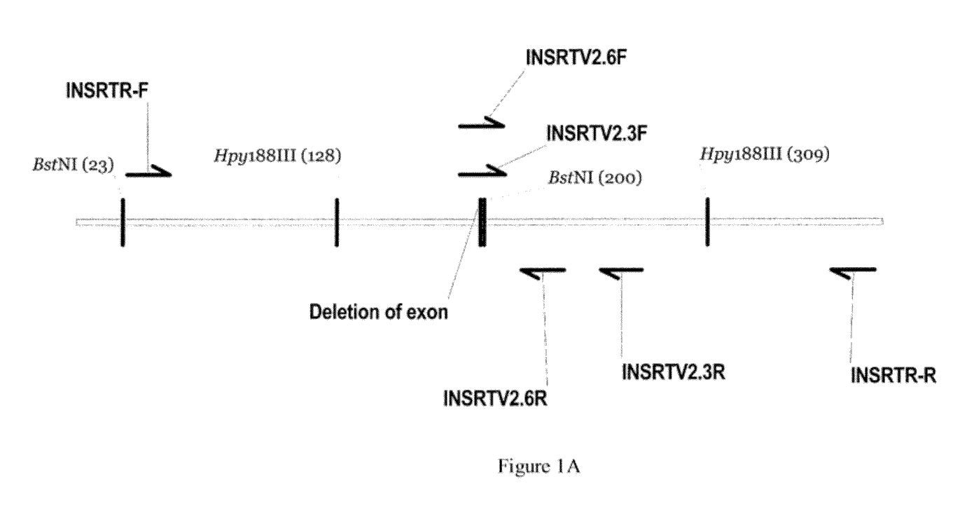

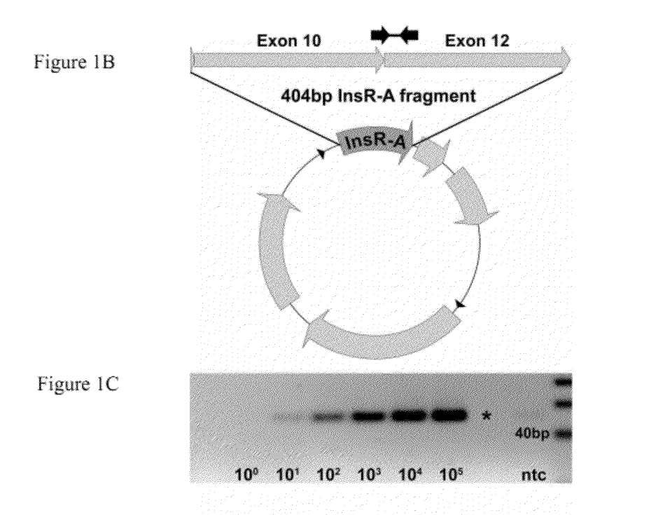

FIG. 1A is a schematic of the insulin receptor alpha sequence showing where a number of different primer sets bind. The sequence of each primer directed toward the insulin receptor alpha sequence is indicated below. FIG. 1B is a schematic showing the IR-A plasmid and the position of binding of primer set #4 within the IR-A region, and FIG. 1C shows the resulting PCR amplification products.

IR-A primer set #4 was designed to amplify a 50 base pair region spanning the junction between Exons 10 and 12. For use as a template, a 404 base pair IR-A nucleic acid sequence corresponding to Exons 10 and 12 of the insulin receptor gene was synthesized and cloned into the pUCminusMCS plasmid vector. Primers were used to amplify purified plasmid using the number of copies of plasmid specified in FIG. 1C and PCR was run for 36 cycles. PCR products were visualized by agarose gel electrophoresis (FIG. 1C).

| | | | Product | SEQ |
|---|---|---|---|---|
| # | Name | Sequence | size | ID NO: |
| 1 | INSR-S(2)-F1 | CGGCGAATGCTGCTCCTGTC | 132 bp | 1 |
| | INSR-S(2)-RU | CGAGATGGCCTGGGGACGAA | | 2 |
| 2 | INSR-L(2)-F2 | GCTGAAGCTGCCCTCGAGGA | 210 bp | 3 |
| | INSR-S(2)-RU | CGAGATGGCCTGGGGACGAA | | 4 |
| 3 | INSR-S2.3-F | GTTTTCGTCCCCAGGCCATCT | 90 bp | 5 |
| | INSR-S2.3-R | GGGGAAAGCTGCCACCGTG | | 6 |
| 4 | INSR-S2.6F | GTTTTCGTCCCCAGGCCATC | 50 bp | 7 |
| | INSR-S2.6R | CCAACATCGCCAAGGGACCT | | 8 |

Insulin Receptor B Primer Sets

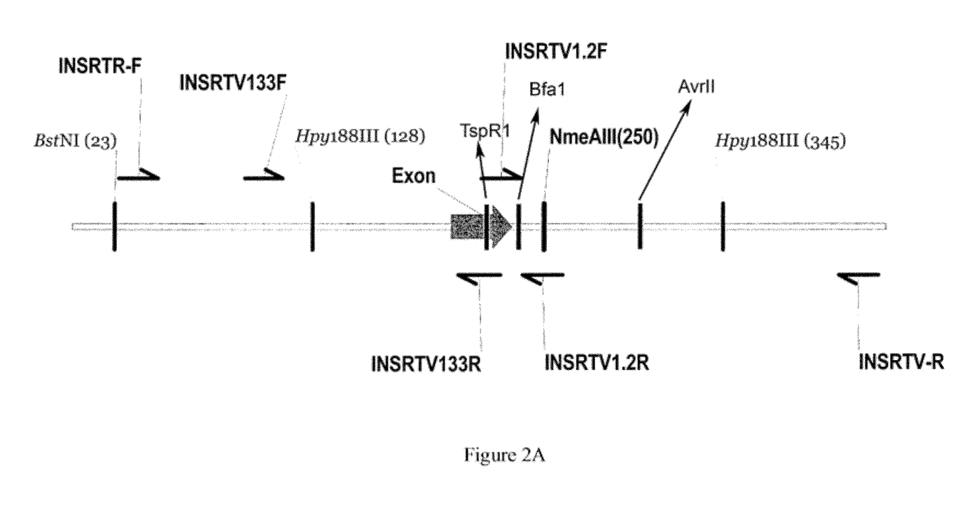

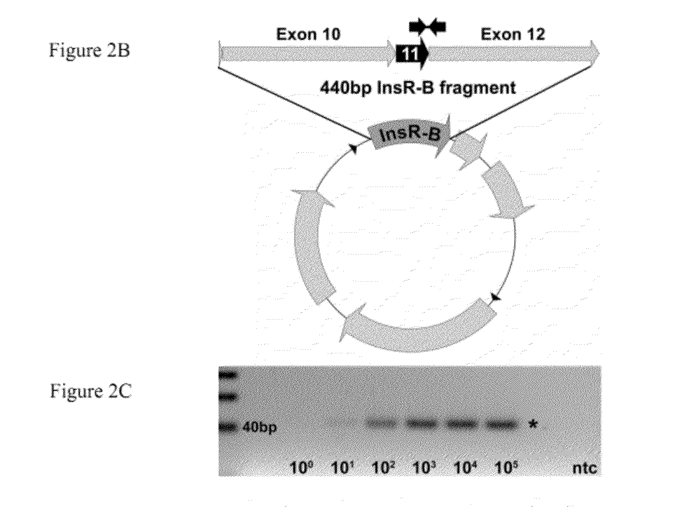

FIG. 2A is a schematic of the insulin receptor beta sequence showing where a number of different primers sets bind. The sequence of each primer directed toward the insulin receptor beta sequence is indicated below. FIG. 2B is a schematic showing the IR-B plasmid and the position of binding of primer set #4 within the IR-B region, and FIG. 2C shows the resulting amplification products.

IR-B primer set #4 was designed to amplify a 42 base pair region spanning the junction between Exons 11 and 12. A 440 base pair IR-B nucleic acid corresponding to Exons 10, 11, and 12 of the insulin receptor gene was synthesized and cloned into the pUCminusMCS plasmid vector. Primers were used to amplify purified plasmid using the number of copies into of plasmid indicated in FIG. 2C and PCR was run for 36 cycles. PCR products were visualized by agarose gel electrophoresis (FIG. 2C).

| | | | Product | SEQ |
|---|---|---|---|---|
| # | Name | Sequence | size | ID NO: |
| 1 | INSR-L(1)-F1 | GCTGAAGCTGCCCTCGAGGA | 223 bp | 9 |
| | INSR-L(1)-R1 | ACCAGTGCCTGAAGAGGTTTT | | 10 |
| 2 | INSR-L(1)-F2 | GTCTCCACCATTCGAGTCTGA | 199 bp | 11 |
| | INSR-L(1)-R2 | ACCAGTGCCTGAAGAGGTTTT | | 12 |
| 3 | INSRTV133F | GAATGCTGCTCCTGTCCAA | 132 bp | 13 |
| | INSRTV133R | ACCAGTGCCTGAAGAGGTTTT | | 14 |
| 4 | INSR-L1.2F | CACTGGTGCCGAGGACCCTA | 42 bp | 15 |
| | INSR-L1.2R | GACCTGCGTTTCCGAGATGG | | 16 |
| 5 | INSRTV1.3 F | CTGCACAACGTGGTTT | 44 bp | 17 |
| | INSRTV1.3 R | GTGCCTGAAGAGGTTTT | | 18 |
| 6 | INSR-L1.4F | TGCCGAGGACCCTAGGCCATC | 51 bp | 19 |
| | INSR-L1.4R | CCAACATCGCCAAGGGACCTG | | 20 |
| 7 | INSR-L1.4F | TGCCGAGGACCCTAGGCCATC | 59 bp | 21 |
| | INSR-L1.4R2 | TCACATTCCCAACATCGCCAA | | 22 |
| 8 | INSR-L1.5F | AGGACCCTAGGCCATCTCGG | 46 bp | 23 |
| | INSR-L1.5R | CCAACATCGCCAAGGGACC | | 24 |
| 9 | INSR-L1.6F | GCACTGGTGCCGAGGACCCTA | 79 bp | 25 |
| | INSR-L1.6R | GGCACGGCCACCGTCACATT | | 26 |
| 10 | INSR-L1.7F | TGCCGAGGACCCTAGGCCAT | 72 bp | 27 |
| | INSR-L1.7R | GGCACGGCCACCGTCACATT | | 28 |
| 11 | INSR-L1.8F | TGCCGAGGACCCTAGGCCAT | 68 bp | 29 |
| | INSR-L1.8R | CGGCCACCGTCACATTCCCA | | 30 |

### Example 3

### Insulin Receptor-Specific Plasmids and Standard Dilutions

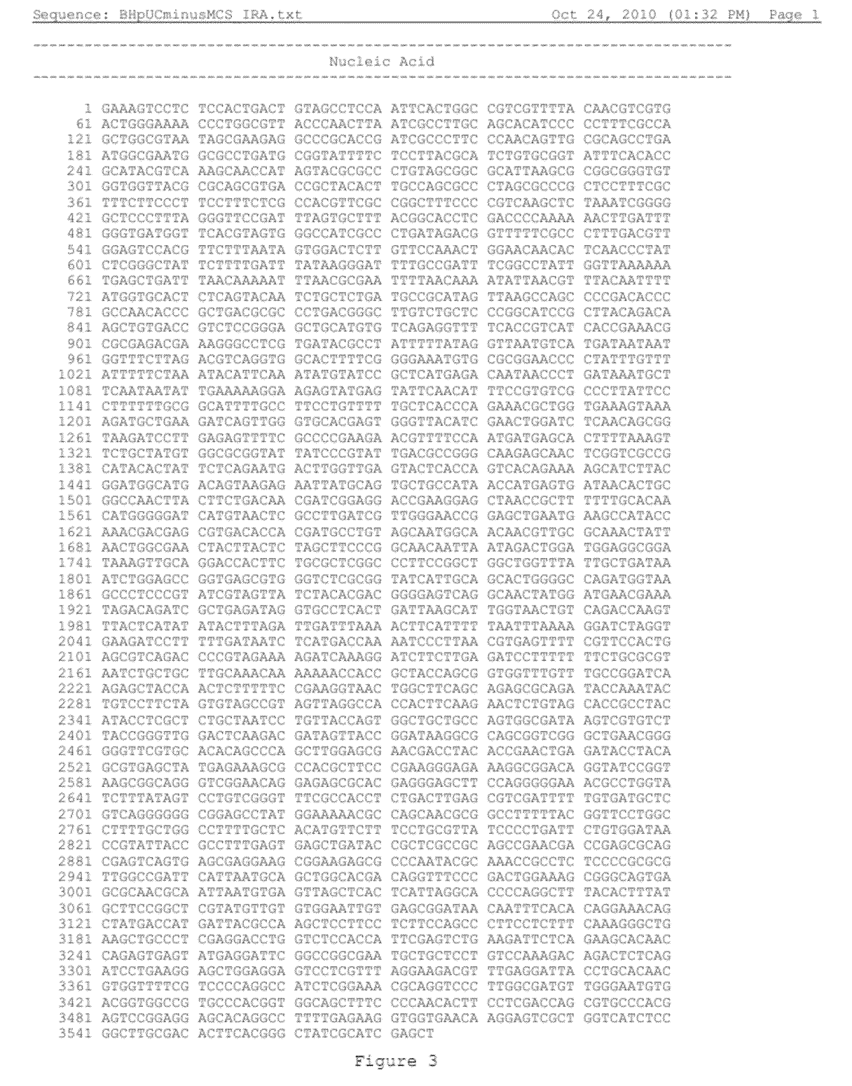

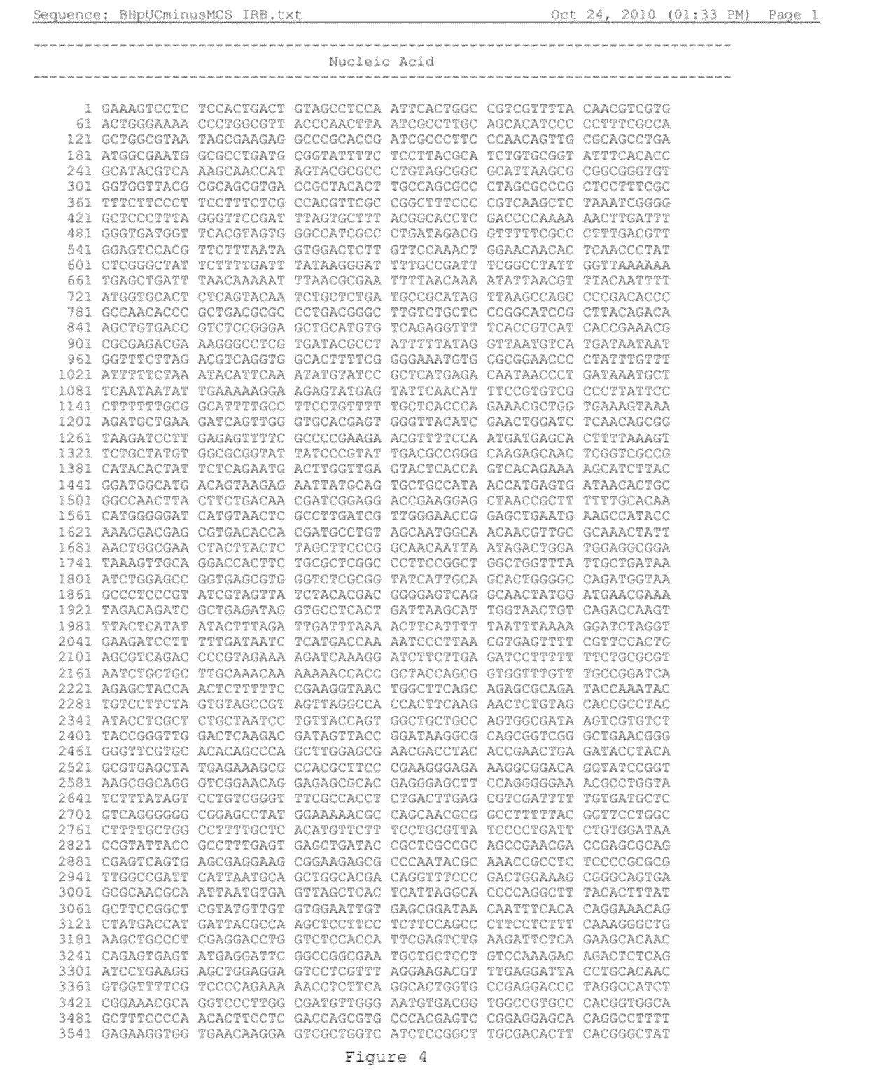

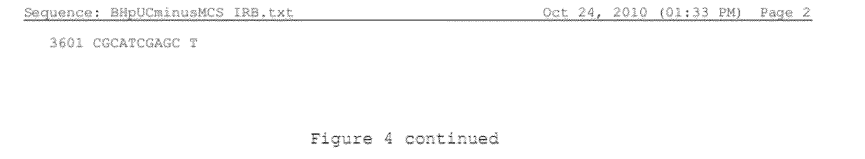

The insulin receptor alpha sequence and the insulin receptor beta sequence were cloned into the Blue Heron pUCminusMCS vector (3171 bp) by Blue Heron (Bothell, Wash.). FIG. 3 shows the nucleic acid sequence of the plasmid containing the insulin receptor alpha sequence (SEQ ID NO:31) and FIG. 4 shows the nucleic acid sequence of the plasmid containing the insulin receptor beta sequence (SEQ ID NO:32).

The characteristics of each vector are shown below.

### Blue Heron Plasmid with Insulin Receptor Alpha (IRA) Insert (BHpUCminusMCS w/ IR-A)

| Size | 3575 bp |
|---|---|
| MW | 2208904.70 |
| μg/ml (ng/μl) | 50.3 |
| g/mol | 2.21E+06 |
| ng/mol | 2.21E+15 |
| copies/ng | 2.73E+08 |
| copies/μl | 1.37E+10 |
| | μl |
| 10¹⁴ molecules | 7294.79 |
| 10¹³ molecules | 729.48 |
| 10¹² molecules | 72.95 |
| 10¹¹ molecules | 7.29 |
| 10¹⁰ molecules | 0.73 |

### Blue Heron Plasmid with Insulin Receptor Beta (IRB) Insert (BHpUCminusMCS w/ IR-B)

| | |
|---|---|
| Size | 3611 |
| MW | 2231150.7 |
| μg/ml (ng/μl) | 50.1 |
| g/mol | 2.23E+06 |
| ng/mol | 2.23E+15 |
| copies/ng | 2.70E+08 |
| copies/μl | 1.35E+10 |
| | μl |
| 10¹⁴ molecules | 7397.67 |
| 10¹³ molecules | 739.77 |
| 10¹² molecules | 73.98 |
| 10¹¹ molecules | 7.40 |
| 10¹⁰ molecules | 0.74 |

### Example 4

### Insulin Receptor Isoform-Specific qPCR

3 μL of IR-specific plasmid standard dilutions, samples, and non-template controls were added to a 384 well plate in triplicate. qPCR Master Mix was assembled with ABI Power SybrGreen 2× Master Mix (Cat. No. 4367659), primers, and water and added to each well containing a sample. Assembled qPCR plates were covered with clear adhesive covers, and centrifuged to remove bubbles before being run in an ABI 7900HT Real Time PCR machine.

Insulin Receptor Alpha Isoform qPCR.

Primers: Pair #4 (INSR-S2.6F and INSR-S2.6R)

Final primer reaction concentrations: 128 nM each

Standard conditions: All reactions were preformed in triplicate. Standard template or sample unknowns made up a maximum of 10% of the total reaction volume. (10 μL reaction would contain 1 μL of DNA in this example)

Standard Template: IRA plasmid was log-fold diluted down to 1 copy per μL in 0.01 M Tris buffer (pH 8.0). 10⁰ thru 10^{6.4} copies were run in triplicate on each plate.

Unknown determination: Assay samples were diluted by 5 with molecular grade water before being run in the qPCR assay.

Reaction Conditions for ABI 7900HT

Denature: 95° C. for 10 minutes

40 Cycles
Denature: 95° C. for 15 sec
Annealing: 60.5° C. for 20 sec
Extension: 72° C. for 20 sec

Melt Curve Analysis
95° C. for 15 sec
60° C. for 15 sec with a 2% ramp in temperature from 60° C. to 95° C.

Insulin Receptor Beta Isoform qPCR.

Primers: Pair #4 (INSR-L1.2F and INSR-L1.2R)

Final primer reaction concentrations: 32 nM each

Standard conditions: All reactions were performed in triplicate. Standard template or sample unknowns made up a maximum of 10% of the total reaction volume

(10 μL reaction would contain 1 μL of DNA in this example)

Standard Template: IRB plasmid was log-fold diluted down to 1 copy per μL in 0.01 M Tris buffer (pH 8.0). 10⁰ thru 10^{6.4} copies were run in triplicate on each plate.

Unknown determination: Assay samples were diluted by 5 with molecular grade water before being run in the qPCR assay.

Reaction Conditions for ABI 7900HT:

Denature: 95° C. for 10 minutes

40 Cycles
Denature: 95° C. for 15 sec
Annealing: 54° C. for 20 sec
Extension: 72° C. for 20 sec

Melt Curve Analysis
95° C. for 15 sec
60° C. for 15 sec with a 2% ramp in temperature from 60° C. to 95° C.

After completion of IR qPCR, standard and sample replicate data were examined for acceptable amplification efficiency (e.g., 95-105%) and for product-specific melt curves. Sample or standard replicates which did not conform were eliminated from the analysis.

Values for conforming expression results were corrected for variances using RPL19 control gene expression. Average replicate values were multiplied by the sample dilution factor before being reported in copies per μg RNA.

The assay platform used was a Sybr Green Quantitative PCR Assay, and the products were quantitated by generating standard curves. The reaction chemistry utilizes Sybr Green dye, which is incorporated into all double-stranded DNA products. Thus, a melt curve was generated for each reaction to ensure a single specific product was generated in each reporting well. Melt curves from unknowns and the standard curve were compared and reactions with aberrant melt curves were eliminated from the analysis.

Validation Table for IR-A Primer Set and IR-B Primer Set

| InsR qPCR Assay | IR-A #4 | IR-B #4 |
|---|---|---|
| Sequences | F-GTTTTCGTCCCCAGGCCATC | F-CACTGGTGCCGAGGACCCTA |
| | (SEQ ID NO: 7) | (SEQ ID NO: 15) |
| | R-CCAACATCGCCAAGGGACCT | R-GACCTGCGTTTCCGAGATGG |
| | (SEQ ID NO: 8) | (SEQ ID NO: 16) |
| Primer | 128 | 32 |
| concentration (nM) | | |
| Annealing temp (C.) | 60.5 | 54 |
| Reaction | 40 Cycles | |
| parameters | Denature: 15 sec | |
| | Annealing: 20 sec | |
| | Extension: 20 sec | |
| | Melt cure analysis from 60 C. to 95 C. | |
| Amplification | 100.71% ± 3.28% | 99.98% ± 2.32% |
| Efficiency | | |
| Ave ± SD, (n = 10) | | |
| Slope | -3.305 ± 0.077 | −3.322 ± 0.054 |
| Ave ± SD, (n = 10) | | |
| Intercept | 36.97 ± 0.170 | 39.88 ± 0.376 |
| Ave ± SD, (n = 10) | | |
| R squared | 0.996 ± 0.003 | 0.975 ± 0.011 |
| Ave ± SD, (n = 10) | | |
| LOD (copies) | 5 | 4 |
| (n = 48) | | |
| Intra-assay | 1.03% ± 0.86% | 0.71% ± 0.55% |
| variation | | |
| Ave ± SD, (n = 10) | | |
| Overall CV | 1.073% ± 0.959% | 3.052% ± 2.6% |
| Ave ± SD, (n = 10) | | |
| Range of Detection | 5-10^{6.4} | 4-10^{6.4} |
| (copies) | | |
| Average Recovery | 94.87 % ± 4.83 % | 97.27 % ± 5.54% |
| Ave ± SEM, (n = 45) | | |
| Isoform | InsR-B to IR-A | InsR-A to IR-B |
| Crossreactivity | 1-2,500,000 | 1-2,500,000 |
| Input Ratios Tested | | |
| Isoform | 0.188% when IR-B | No |
| Crossreactivity | plasmid ≧ 10^{4.4} copies per | |
| Detected | reaction | |
| Ratio | | |
| FFPE Breast Tumor | 93.2% | 91.8% |
| Sample Detection | (192/206) | (189/206) |
| Rate | | |

### Example 5

### In Vitro Results

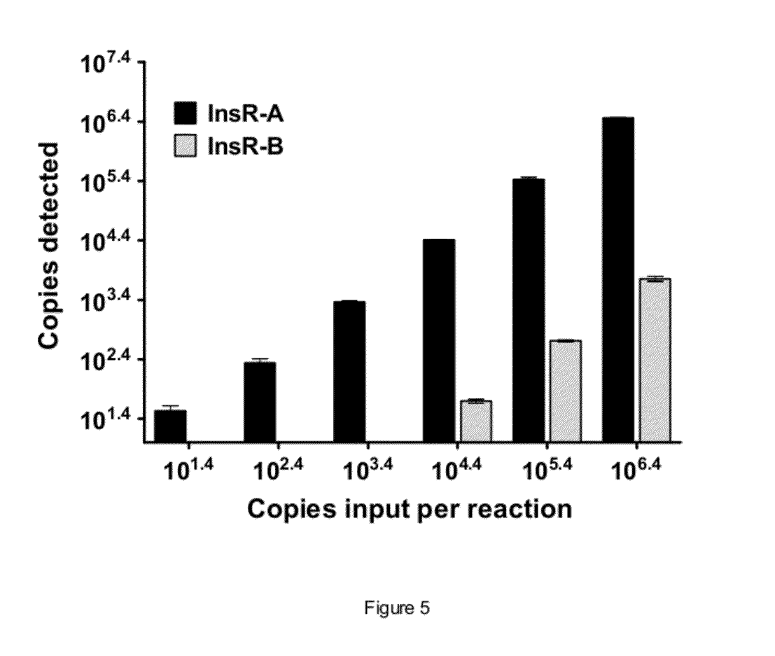

FIG. 5 is a graph that demonstrates the specificity of IR-A primer set #4 for IR-A plasmid and the cross-reactivity of IR-A primer set #4 with the IR-B plasmid. Data are graphed as geometric mean±95% CI. The IR-A PCR assay successfully detected InsR-A plasmid diluted across a 6-log range. IR-B plasmid was diluted across the same input range to determine if IR-A primer set #4 exhibited any crossreactivity with the IR-B plasmid. The IR-A PCR primer set #4 exhibited an 0.188% cross-reactivity when 10^{4.4} or more copies of the IR-B plasmid were input into an IR-A PCR reaction; this cross-reactivity could be eliminated through dilution as lower IR-B plasmid inputs failed to cross-react.

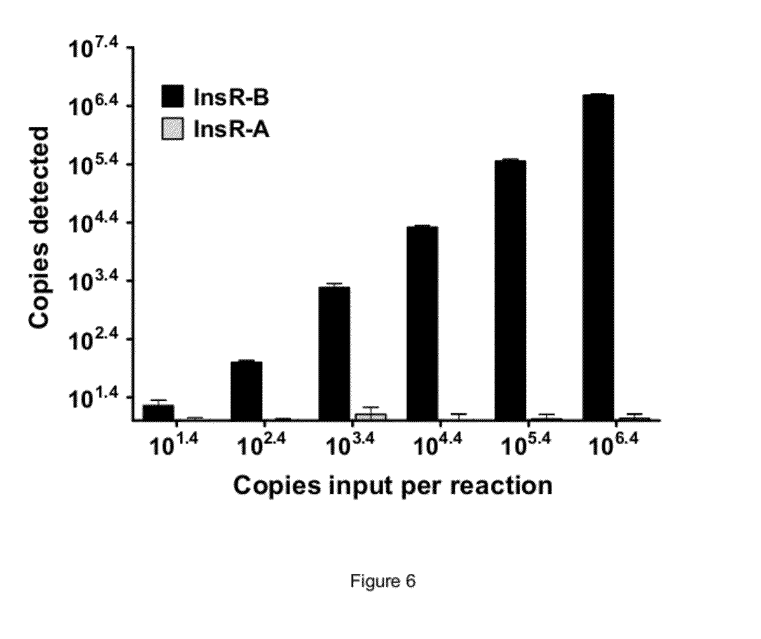

FIG. 6 is a graph that demonstrates the specificity of IR-B primer set #4 for IR-B plasmid and the cross-reactivity of IR-B primer set #4 with the IR-A plasmid. Data are graphed as geometric mean±95% CI. The IR-B PCR assay successfully detected InsR-A plasmid diluted across a 6-log range. IR-A plasmid was diluted across the same input range to determine if IR-B primer set #4 exhibited any cross-reactivity with the IR-A plasmid. The IR-B primer set #4 did not cross-react with the IR-A plasmid at any level of input tested.

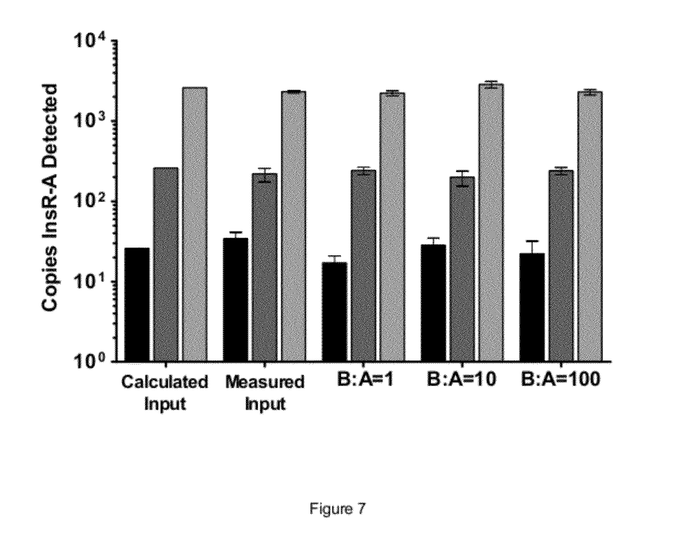

FIG. 7 is a graph that shows the detection of IR-A plasmid in a high background of IR-B plasmid using IR-A primer set #4. Calculated input bars for each assay are theoretical values to confirm measured input values of 10^{1.2} (black), 10^{2.2} (medium grey), and 10^{3.2} (light grey) copies of IR-A plasmid per reaction. Each measured input value was tested in combination with IR-B plasmid using IR-B:IR-A ratios of 1, 10, and 100 copies per reaction. Measured input and recovery input ratio data are graphed as geometric mean±95% CI. The average recovery of all the input target dilutions was 94.87%±4.88%. (IR-B to IR-A input ratio=B:A).

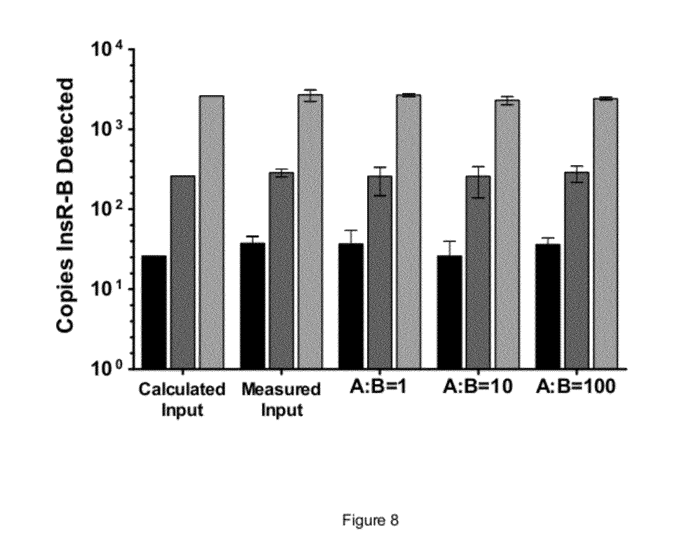

FIG. 8 is a graph that shows the detection of IR-B plasmid in a high background of IR-A plasmid using IR-B primer set #4. Calculated input bars for each assay are theoretical values to confirm measured input values of 10^{1.2} (black), 10^{2.2} (medium grey), and 10^{3.2} (light grey) copies of IR-B plasmid per reaction. Each measured input value was tested in combination with IR-A plasmid using IR-A:IR-B ratios of 1, 10, and 100 copies per reaction. Measured input and recovery input ratio data are graphed as geometric mean±95% CI. The average recovery of all the input target dilutions was 97.27%±5.6%. (IR-A to IR-B input ratio=A:B).

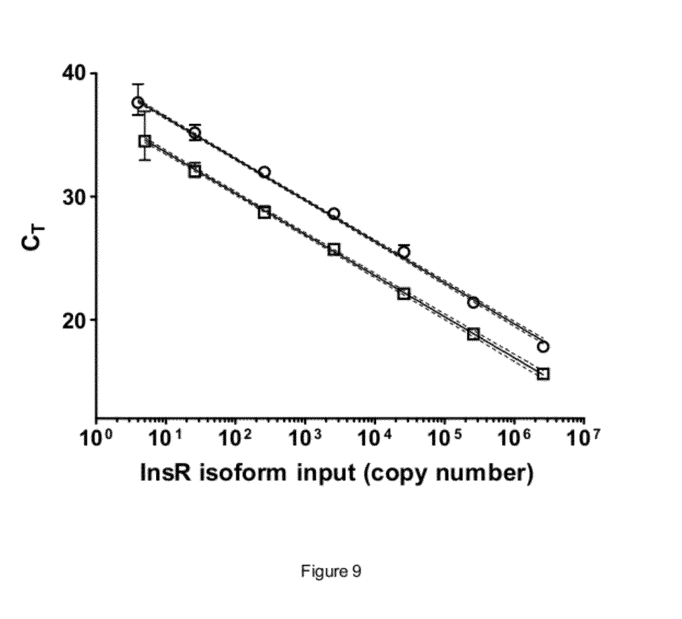

FIG. 9 is a graph that shows the limits of detection (LOD) for IR-A primer set #4 and IR-B primer set #4. Each line represents combined data from 8 separate assays. Each assay was composed of a 7-point standard curve with each point run in triplicate (unless otherwise noted). The grand mean and SD of all assays were used to generate a best fit line and a 95% confidence interval for each standard curve. The LOD was tested between 0 and 10 copies per reaction using 6 replicates for each input copy number tested. The LOD of each assay was determined empirically using the lowest input copy number at which 95% of assay replicates (n=48) reported target specific melt curves. The LOD for IR-A (circle) and IR-B (square) primer set were 5 and 4 copies, respectively.

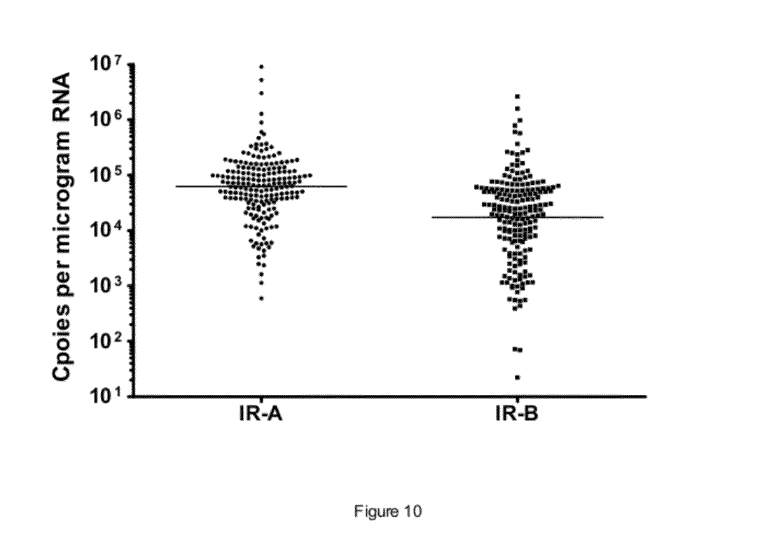

FIG. 10 demonstrates the successful quantification of insulin receptor isoform alpha and beta in FFPE tumors using IR-A primer set #4 and IR-B primer set #4. Scatter plots are shown of IR-A and IR-B expression measured from 206 clinically-archived FFPE tumors. Prior to being graphed, all expression data were normalized to RNA input and expression of a control gene. Lines denote geometric means for each data set. IR-A primer set #4 was able to successfully quantify 93.2% (192 positives out of 206 total) of FFPE tumors samples tested, and IR-B primer set #4 was able to successfully quantify 91.8% (189 positives out of 206 total) of FFPE tumors samples tested.

### Example 6

### In Vivo Experiments

Human breast cancer xenograft tumor tissue harvested from mice (n=5), human ovarian cancer xenograft tumor tissue harvested from mice (n=3), pooled human brain cancer xenograft tissue harvested from mice, and normal brain tissue harvested from mice were used in qPCR reactions using the primers and the conditions described in the Examples above. The results are shown below in Tables 1-4.

TABLE 1
Human breast cancer xenograft tumor IR expression

| | Average # of copies of IR per ng RNA | +SD | −SD |
|---|---|---|---|
| IRA | 12340.01 | 3698.12 | 2845.39 |
| IRB | 679.83 | 391.08 | 248.26 |

TABLE 2
Human ovarian cancer xenograft tumor IR expression

| | Average # of copies of IR per ng RNA | +SD | −SD |
|---|---|---|---|
| IRA | 2474.57 | 7.09 | 7.07 |
| IRB | 196.55 | 28.36 | 24.78 |

TABLE 3
Pooled human brain cancer xenograft tissue

| | Average # of copies of IR per ng of RNA | +SD |
|---|---|---|
| IRA | 3180.94 | 1105.08 |
| IRB | 252.93 | 31.81 |

TABLE 4
Mouse normal brain tissue

| | Average # of copies of IR per ng of RNA | +SD |
|---|---|---|
| IRA | Not detected | |
| IRB | Not detected | |

Based on the results in Tables 1-4, human IRA and IRB can be quantitatively detected in biological tissues, including tumor tissues. Also based on the results in Tables 1-4, IRA is expressed at higher levels than IRB in the xenograft tumors examined.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of determining the amount of insulin receptor alpha (IR-A) in a sample, comprising the steps of:
a) amplifying an IR-A sequence in a sample using a pair of IR-A primers to produce an IR-A amplification product, wherein the pair of IR-A primers is selected from the group consisting of SEQ ID NOs: 1 and 2; SEQ ID NOs: 3 and 4; SEQ ID NOs: 5 and 6; and SEQ ID NOs: 7 and 8; and
b) determining the amount of the IR-A amplification product.

2. The method of claim 1, wherein the sample is a biopsy.

3. The method of claim 2, wherein the biopsy is a tumor biopsy.

4. The method of claim 1, wherein the sample is from a patient that has cancer.

5. The method of claim 1, wherein the pair of primers is SEQ ID NOs:7 and 8.

6. The method of claim 1, wherein the determining step comprises using at least one labeled IR-A probe.

7. A method of detecting the amount of insulin receptor beta (IR-B) in a sample, comprising the steps of:
a) amplifying an IR-B sequence in a sample using a pair of IR-B primers to produce an IR-B amplification product, wherein said pair of IR-B primers is selected from the group consisting of SEQ ID NOs: 9 and 10; SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; and SEQ ID NOs: 29 and 30; and
b) determining the amount of the IR-B amplification product.

8. The method of claim 7, wherein the sample is a biopsy.

9. The method of claim 8, wherein the biopsy is a tumor biopsy.

10. The method of claim 7, wherein the sample is from a patient that has cancer.

11. The method of claim 7, wherein the pair of primers is SEQ ID NOs:15 and 16.

12. The method of claim 7, wherein the determining step comprises using at least one labeled IR-B probe.

13. An article of manufacture, comprising:
(a) a pair of primers for detecting the amount of insulin receptor alpha (IR-A) in a sample, wherein the pair of IR-A primers is selected from the group consisting of SEQ ID NOs: 1 and 2; SEQ ID NOs: 3 and 4; SEQ ID NOs: 5 and 6; and SEQ ID NOs: 7 and 8; or
(b) a pair of primers for detecting the amount of insulin receptor beta (IR-B) in a sample, wherein the pair of IR-B primers is selected from the group consisting of SEQ ID NOs: 9 and 10; SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; and SEQ ID NOs: 29 and 30; or
(c) a pair of IR-A primers from (a) and a pair of IR-B primers from (b).

14. The article of manufacture of claim 13, wherein the pair of IR-A primers is SEQ ID NOs: 7 and 8.

15. The article of manufacture of claim 13, wherein the pair of IR-B primers is SEQ ID NOs: 15 and 16.

16. The article of manufacture of claim 13, further comprising a pair of IR-A probes.

17. The article of manufacture of claim 13, further comprising a pair of IR-B probes.
